(19) United States
(12) Patent Application Publication
(10) Pub. No.: US 2016/0095721 A1
(43) Pub. Date: Apr. 7, 2016

(54) TRANS-OSSEOUS OBLIQUE LUMBOSACRAL FUSION SYSTEM AND METHOD

(71) Applicant: Quandary Medical LLC, Denver, CO (US)

(72) Inventors: Gerald Russell Schell, Denver, CO (US); Jeffrey R. Schell, Denver, CO (US)

(73) Assignee: QUANDARY MEDICAL LLC, Denver, CO (US)

(21) Appl. No.: 14/875,460

(22) Filed: Oct. 5, 2015

Related U.S. Application Data
(60) Provisional application No. 62/059,892, filed on Oct. 4, 2014.

(51) Int. Cl. A61F 2/46; A61B 17/70; A61B 17/16; A61F 2/44

(52) U.S. Cl. CPC A61F 2/4611; A61F 2/4455; A61B 17/7094; A61B 17/1664; A61B 2017/00022

(57) ABSTRACT

Embodiments of the invention are directed to a method of directing medical instruments towards an intervertebral disc space, removing intervertebral disc material from such disc space, and filling such disc space with a material to fuse vertebrae. Embodiments of the invention are further directed to a path that passes structural portions of an ilium. In some aspects, embodiments of the invention are directed to using electro stimulation to avoid certain nerve roots to access an L5-S1 disc space for the purposes of bone fusion.

### CROSS-REFERENCE TO RELATED APPLICATIONS

This application is a non-provisional of, and claims the benefit under 35 U.S.C. §120 of U.S. Provisional Application No. 62/059,892 entitled “Trans-Osseous Oblique Lumbosacral Fusion System and Method” filed on Oct. 4, 2014, which is hereby incorporated by reference in its entirety.

### FIELD OF THE INVENTION

The field relates generally to a medical procedure, and more particularly to a medical process for a trans osseous oblique lumbar interbody fusion.

### BACKGROUND

The spine comprises a number of vertebrae separated by intervertebral discs. There are 24 articulating vertebrae, including the cervical, thoracic, and lumbar bones, as well as 9 fused bones associated with the sacrum or the coccyx. The intervertebral discs, having a nucleus pulposus and surrounded by annulus fibrosus, act as shock-absorbing layers between the articulating vertebrae, and helps to distribute pressure associated with movement of the spine.

Mechanical stress of the spine can lead to intervertebral disc degeneration and spinal factures. A number of mechanical factors, such as injuries that lead to compressive, bending, and compressive/bending stresses of the vertebra or intervertebral discs, can lead to spinal degeneration and pain associated with the spine. In addition, ageing is associated with dehydration of the nucleus pulposus, resulting in a less effective shock absorption and degradation of the intervertebral disc. Spondylolisthesis, or the forward displacement of the vertebra, can commonly occur in the fifth lumbar vertebra.

Spinal fusion procedures help to secure two or more vertebrae. Spinal fusions stabilize the vertebral column, mitigate damage to the spinal cord, and decrease pain associated with spinal degeneration and spinal ageing. In general, spinal fusion procedures include methods related to accessing the intervertebral disc space, removing the nucleus pulposus and annulus fibrosus, and filling the intervertebral space with an implant embedded in autograft or allograft bone matter to fuse the two vertebrae. The implant, in general, provides stability between two vertebrae as to prevent collapse of the intervertebral disc space during healing, while the autograft or allograft bone matter fuses said vertebrae. In addition, a vertebrae fixation procedure, wherein braces are introduced to the exterior surface of the vertebral column often accompanies the spinal fusion procedure to increase stability and facilitate bone fusion between the vertebrae.

A L5-S1 spinal fusion is a procedure performed for stabilizing and reducing pain of the joint between the fifth lumbar vertebra (L5) and sacral bone (S1). A number of procedures are used to access the L5-S1 intervertebral disc space for spinal fusions. These procedures include approaching the spine from the anterior side, such as in Anterior Lumbar Interbody Fusion (ALIF), the posterior side, such as in Posterior Lumbar Interbody Fusion (PLIF) and the posterolateral side, such as in Transforaminal Lumbar Interbody Fusion (TLIF). However, a number of disadvantages exist for these various approaches.

For instance, an anterior approach to the L5-S1 intervertebral disc through the peritoneum, such as in ALIF, increases the risk of damage to organs and the circulatory system. In addition, an anterior approach has an increased risk of retrograde ejaculation in males, due to potential damage to the superior hypogastric plexus. A posterior approach, such as in PLIF and TLIF, has an increased risk of damage to posterior paraspinal muscles and ligaments. Both PLIF and TLIF additionally involve a laminectomy to access the intervertebral disc. While TLIF involves less removal of soft tissue and muscle due to a slightly lateral approach, in general, intervertebral disc material may not be completely removed as compared with PLIF.

Some of the challenges involved with accessing the L5-S1 intervertebral disc space from the lateral side include identifying a minimally invasive access angle. A number of approaches are found in the prior art, such as for example, a transiliac-transsacral method as disclosed by Spineology (U.S. Pat. No. 8,439,925). However, such transiliac-transsacral method is not entirely efficient, as such method discloses an approach channel that penetrates multiple bone structures, including the iliac ala, sacroiliac joint, and sacral ala. Furthermore, such prior art patent requires the use of a guide instrument including a curvilinear frame, wherein such guide instrument is additionally secured to an existing pedicle bone structure. These inefficiencies can lead to increased complexity of the surgery and time spent on the surgery.

### BRIEF DESCRIPTION

In general, embodiments of the invention are related to a medical procedure to accomplish a spinal fusion. More particularly, embodiments are related to a spinal fusion at the L5-S1 intervertebral disc space. The preferred embodiment of the invention solves some of the problems in prior art processes associated with accessing the L5-S1 intervertebral disc space by a novel mode of entry and associated steps. The preferred embodiment of the invention is related to the removal of intervertebral disc matter through the iliac bone for a safer, minimally invasive spinal fusion process.

### BRIEF DESCRIPTION OF THE DRAWINGS

FIG. 1. Illustration of a trans-osseous oblique lateral fusion approach embodiment in relation to the pelvic bones and lumbar spine in a three quarters view.

FIG. 2A. Illustration of the trans-osseous oblique lateral fusion approach embodiment in relation to the pelvic bones and spine from a lateral view.

FIG. 2B. Illustration of the trans-osseous oblique lateral fusion approach embodiment in relation to the pelvic bones and spine from a lateral view, where the right ilium is transparently shown.

FIG. 2C. Illustration of the trans-osseous oblique lateral fusion approach embodiment in relation to the pelvic bones and spine from the lateral view, where a right ilium is not shown.

FIG. 3. Illustration of a trans-osseous oblique lateral fusion approach embodiment in relation to the pelvic bones and spine from a posterior view

FIG. 4. An exemplary flow diagram of the trans-osseous oblique lateral fusion approach embodiment.

FIG. 5. Illustration of the trans-osseous oblique lateral fusion approach embodiment showing passage through Kambin's triangle in a lateral view, in one embodiment of the invention.

FIG. 6A. Illustration of the trans-osseous oblique lateral fusion approach embodiment showing the approximate passage of a guide wire to the L5-S1 intervertebral disc space from the top-down view, in one embodiment of the invention.

FIG. 6B. Illustration of the trans-osseous oblique lateral fusion approach embodiment showing the approximate passage of a disc drill to the L5-S1 intervertebral disc space from the top-down view, in one embodiment of the invention.

FIG. 7A. Illustration of the trans-osseous oblique lateral fusion approach embodiment showing the approximate passage of the bone drill through the left iliac crest to open the transiliac aperture from the top-down view, in one embodiment of the invention.

FIG. 7B. Illustration of the trans-osseous oblique lateral fusion approach embodiment showing the approximate passage of the access portal to the L5-S1 intervertebral disc space from the top-down view, in one embodiment of the invention.

### NUMERICAL REFERENCES

1. Lumbosacral (L5-S1) intervertebral disc representation
2. 5^{th} Lumbar vertebra representation
3. Sacrum including the first through fifth Sacral vertebrae representation
4. L4-L5 intervertebral disc representation
5. 4^{th} Lumbar vertebra representation
6. L3-L4 intervertebral disc representation
7. 3^{rd} Lumbar vertebra representation
8. Right ilium representation
9. transiliac aperture in the preferred embodiment of the invention
10. Medical instruments for accessing the L5-S1 intervertebral disc in the preferred embodiment of the invention
11. Coccyx representation
12. L2-L3 intervertebral disc representation
13. 2^{nd} Lumbar vertebra representation
14. L1-L2 intervertebral disc representation
15. 1^{st} Lumbar vertebra representation
16. T12-L1 intervertebral disc representation
17. 12^{th} Thoracic vertebra representation
18. Left Ilium representation
19. Determine Approach Angle step in the preferred embodiment of the invention
20. Skin Incision step in the preferred embodiment of the invention
21. Calibrate Ilium Approach step in the preferred embodiment of the invention
22. Insert Jamshidi Needle step in the preferred embodiment of the invention
23. Needle Imaging step in the preferred embodiment of the invention
24. Modify Needle Path step in the preferred embodiment of the invention
25. Calibrate Kambin Approach step in the preferred embodiment of the invention
26. Insert Stimulating Electrode step in the preferred embodiment of the invention
27. Nerve Conduction step in the preferred embodiment of the invention
28. Neurodiganosis step in the preferred embodiment of the invention
29. Adjust Stimulating Electrode step in the preferred embodiment of the invention
30. Establish Approach Path step in the preferred embodiment of the invention
31. Insert Wire to Disc Space Step in the preferred embodiment of the invention
32. Drill to Disc Space with Wire Drill step in the preferred embodiment of the invention
33. Drill Through Ilium step in the preferred embodiment of the invention
34. Expand Path to Disc Space with Dilators step in the preferred embodiment of the invention
35. Deliver Access Portal step in the preferred embodiment of the invention
36. Intervertebral Disc Removal step in the preferred embodiment of the invention
37. Penetrate Intervertebral Disc with Disc Drill step in the preferred embodiment of the invention
38. Open Disc Space with Disc Shaper step in the preferred embodiment of the invention
39. Remove Disc material with Rongeur step in the preferred embodiment of the invention
40. Open Disc Space with Disc Cutter step in the preferred embodiment of the invention
41. Irrigate and Clean Disc Space step in the preferred embodiment of the invention
42. Intervertebral Disc Space Implantation step in the preferred embodiment of the invention
43. Insert Guide Wire step in the preferred embodiment of the invention
44. Insert Trial Implant step in the preferred embodiment of the invention
45. Pack Graft Material step in the preferred embodiment of the invention
46. Insert Implant step in the preferred embodiment of the invention
47. Verify Implant Position step in the preferred embodiment of the invention
48. Obtain Graft Bone and Prepare for Bone Fusion step in the preferred embodiment of the invention
49. Pack Implant step in the preferred embodiment of the invention
50. Percutaneous Posterior Fixation Procedure found in the prior art.
51. Annulus Fibrosis representation
52. Nucleus Pulposus representation
53. (Omitted)
54. (Omitted)
55. Location of an exemplary right L5-S1 Kambin's Triangle
56. (Omitted)
57. (Omitted)
58. Pubic Symphysis
59. guide wire embodiment
60. wire drill embodiment
61. bone drill embodiment
62. access portal embodiment

### SUMMARY

In one aspect, embodiments of the invention are directed to a method of directing medical instruments towards an intervertebral disc space, removing intervertebral disc material from such disc space, and filling such disc space with a material to fuse vertebrae. In another aspect, embodiments of the invention are directed to a path that passes a portion of an ilium. In another aspect, embodiments of the invention are directed to a method to allow a path from an exterior portion of a body to a disc space that traverses an ilium using electro stimulation to allow a path avoiding certain nerve roots.

### DETAILED DESCRIPTION

In the preferred embodiment of the invention, a trans-osseous oblique lateral fusion approach or a TOOLIF approach, is related to a medical procedure to fuse two vertebrae, by accessing a vertebrae or space between vertebrae by traversing the ilium at an oblique angle. In certain embodiments of a TOOLIF approach, a lumbrosacral joint, including the disc space located between the 5^{th} lumbar vertebra and the sacrum, is accessed through either the left 18 or right 8 ilium, in the preferred embodiment of the invention. Together, in the preferred embodiment of the invention, the trans-osseous oblique lateral fusion approach enables a minimally invasive approach for the fusion of the 5^{th} lumbar vertebra 2 and the sacrum bone 3.

The steps that include the TOOLIF approach enable a medical practitioner to access the L5-S1 intervertebral disc 1 space in a minimally invasive manner. Typical fusion approaches found in the prior art have a number of disadvantages that the TOOLIF approach aims to solve. For instance, in the ALIF procedure, generally having an anterior approach to the L5-S1 intervertebral disc 1 through the peritoneum has an increased risk of damage to organs and the circulatory system, and an increased risk of retrograde ejaculation in males due to potential damage to the superior hypogastric plexus. A posterior approach, such as in PLIF and TLIF, has an increased risk of damage to posterior paraspinal muscles and ligaments. Both PLIF and TLIF may also involve a laminectomy to access the intervertebral disc. By approaching the L5-S1 intervertebral disc 1 through an oblique angle through only the iliac bone, the TOOLIF approach reduces risks involved with other approaches.

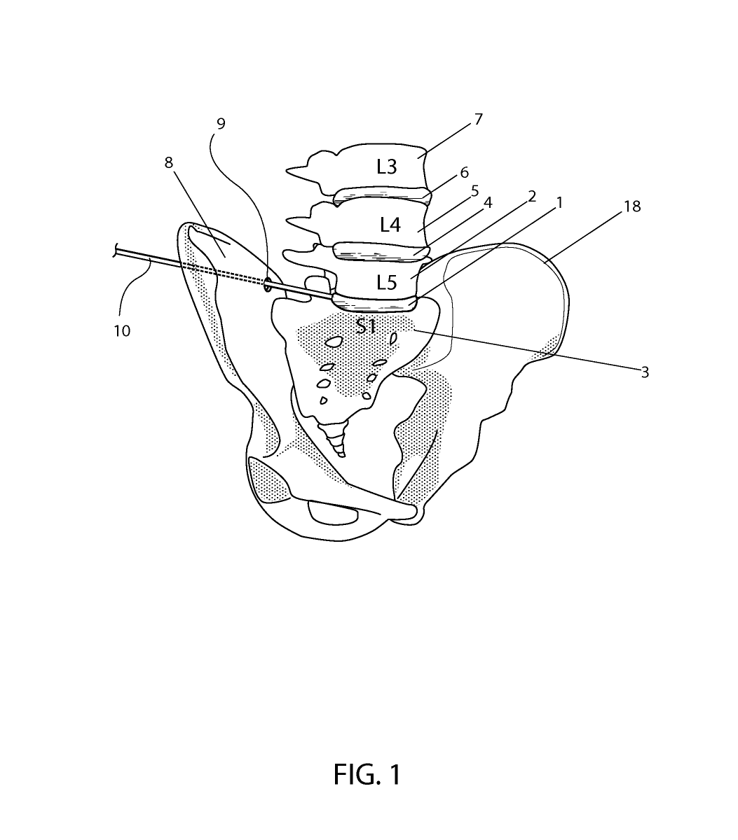

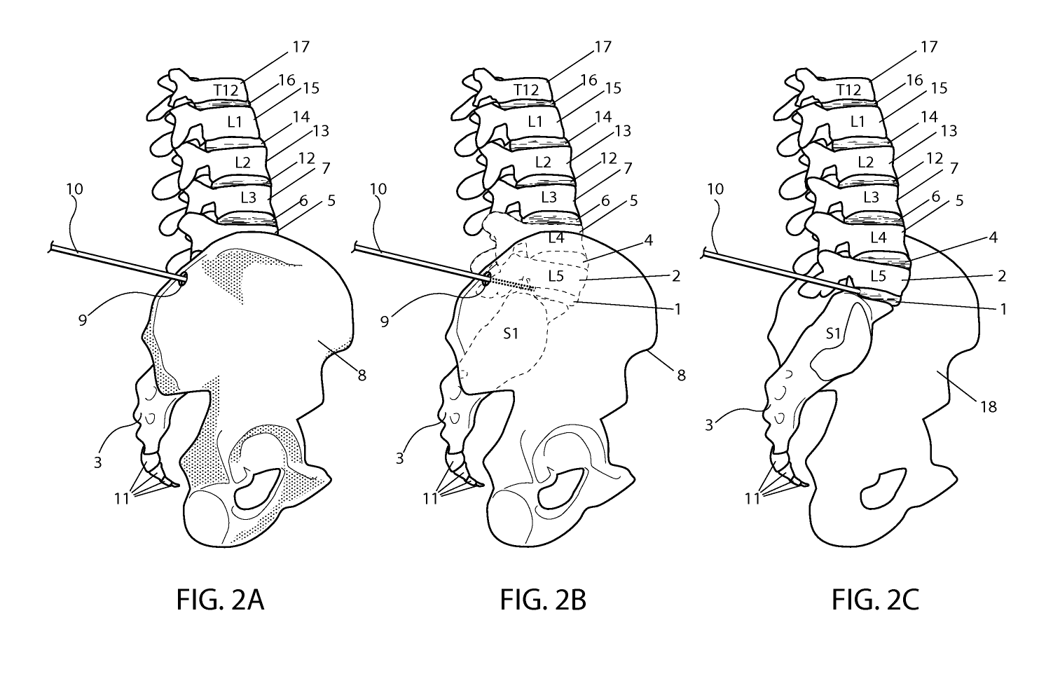

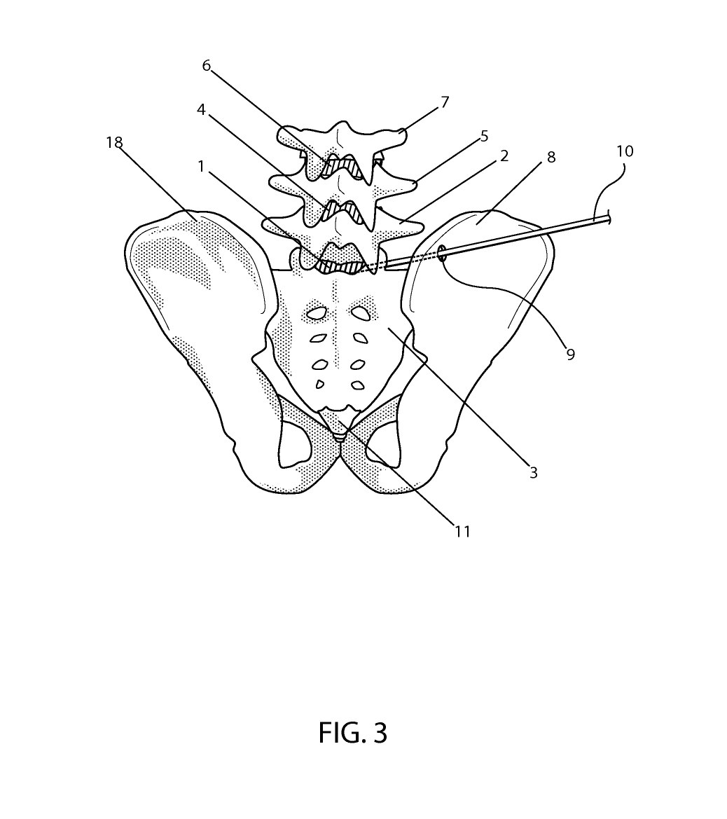

As illustrated in a representative spinal approach of the trans-osseous oblique lateral fusion approach in FIG. 1, FIG. 2 and FIG. 3, the area of the preferred embodiment of the invention takes place in the general vicinity of the pelvis. The coccyx (tailbone) 11, the 12^{th} thoracic vertebra 17, the 1^{st} lumbar vertebra 15, the 2^{nd} lumbar vertebra 13, the 3^{rd} lumbar vertebra 7, the 4^{th} lumbar vertebra 5, the T12-L1 intervertebral disc 16, the L1-L2 intervertebral disc 14, the L2-L3 intervertebral disc 12, the L3-L4 intervertebral disc 6, and the L4-L5 intervertebral disc 4 are shown for visual reference in FIG. 1, FIG. 2 and FIG. 3.

As illustrated in FIG. 1, FIG. 2, and FIG. 3, in an embodiment of the invention, the lumbrosacral intervertebral disc space 1 is accessed by a medical practitioner by traversing either the right ilium 8 or left ilium 18 through a transiliac passage. A transiliac passage, in certain embodiments of the invention, is a path created during the trans-osseous oblique lateral fusion approach in the preferred embodiment of the invention. Certain medical instruments 10 for accessing the L5-S1 intervertebral disc 1, in the preferred embodiment of the invention, includes a number of surgical devices known to persons having ordinary skill in the art related to spinal fusions, including, but not limited to syringes, trephine needles such as Jamshidi needles, cannulae, endoscopes, guide wires, electrotransmitting probes, wire drills, dilators, tubes, disc drills, disc shapers, disc cutters, rongeurs, curettes, and implants. Medical instruments used in the preferred embodiment of the TOOLIF approach are described herein, but are not meant to be limiting in scope, and other embodiments of the invention includes medical instruments described herein and others, without taking away from the essence of the preferred embodiment of the invention.

In general, various aspects of the preferred embodiment of the invention are performed by a medical practitioner, including a number of entities related to a surgical procedure, including but not limited to surgeons, physician's assistants, nurses, technicians, neurodiagnostic technicians, and anesthesiologists. In general, a patient refers to an entity or plurality of entities receiving the TOOLIF approach as disclosed. In the preferred embodiment of the invention, the TOOLIF approach is performed in conjunction with a number of instruments, including, but not limited to bone-imaging or bone scanning devices such as, for example, biplanar fluoroscopes (also referred to as C-Arm Fluoroscopes), and electro stimulator and related accessories. In the preferred embodiment of the invention, a bone-imaging or bone scanning devices captures images of the patient through various views, including but not limited to the lateral view and anterior-posterior (AP) view of the patient.

In the preferred embodiment of the invention, the TOOLIF approach includes a number of steps to ensure that an implant device traverses the ilium and past certain nerves into the L5-S1 intervertebral disc 1 space at an angle that is parallel to the plane of an intervertebral disc space. A transiliac passage that is parallel to the plane of the intervertebral disc space is effectively the key feature of the preferred embodiment of the invention. By establishing an ideal transiliac passage, further including an approach path and an approach angle that is parallel to the plane of the intervertebral disc, the preferred embodiment of the invention provides a number of advantages including a maximal space for access to the intervertebral disc contents, removal of such contents, and placement of implant and fusion material, while providing an overall safer and faster spinal fusion approach. Furthermore, traversing the ilium in the preferred embodiment of the invention is an additional key feature to establish approach path and approach angle that is parallel to the plane of the intervertebral disc. Together, the preferred embodiment of the TOOLIF approach enables a minimally invasive procedure for fusing the L5-S1 vertebral discs.

Another key aspect of the preferred embodiment of the invention is that the approach path and approach angle to the intervertebral disc space is established prior to drilling the transiliac aperture 9. A transiliac aperture, in certain embodiments, is an opening created on the iliac crest. The transiliac aperture, in the preferred embodiment of the invention is created during the Drill Through Ilium step 33, and accommodates medical instruments having a large diameter, including medical instruments 10 involved in the Expand Path to Disc Space with Dilators 34 step, Deliver Access Portal 35 step, Intervertebral Disc Removal 36 step, and the Intervertebral Disc Space Implantation 42 steps. In general, in the preferred embodiment of the invention, the transiliac passage traverses Kambin's Triangle without damaging the nerves associated with Kambin's Triangle.

The transiliac aperture is created only once the approach path and approach angle of the transiliac passage to the intervertebral disc is established. The inventor has discovered that identifying the ideal transiliac passage prior to creating the transiliac aperture 9 is an important aspect of the preferred embodiment of the invention. By identifying the ideal transiliac passage prior to creating the transiliac aperture 9, the medical practitioner avoids unnecessary drilling of bone and tissue. Moreover, establishing the ideal transiliac passage effectively ensures that important nerve roots, such as those related to Kambin's Triangle, are avoided and not damaged. The preferred embodiment of procedure herein describes the steps necessary to establish an ideal transiliac passage prior to creating a transiliac aperture 9, thereby effectively performing a minimally invasive spinal fusion procedure.

In the preferred embodiment of the invention, the general process of the TOOLIF approach occurs after anesthesia, disinfection, and other standard procedures and practices related to surgery and/or spinal fusions known to persons having ordinary skill in the art. Due to the minimally invasive nature and relatively straightforward nature of the TOOLIF approach, it is in general recognized by the inventor that the patient may remain conscious during the length of time of such approach in the preferred embodiment of the invention. In the preferred embodiment of the invention, the TOOLIF approach is performed on a patient laying in a prone position.

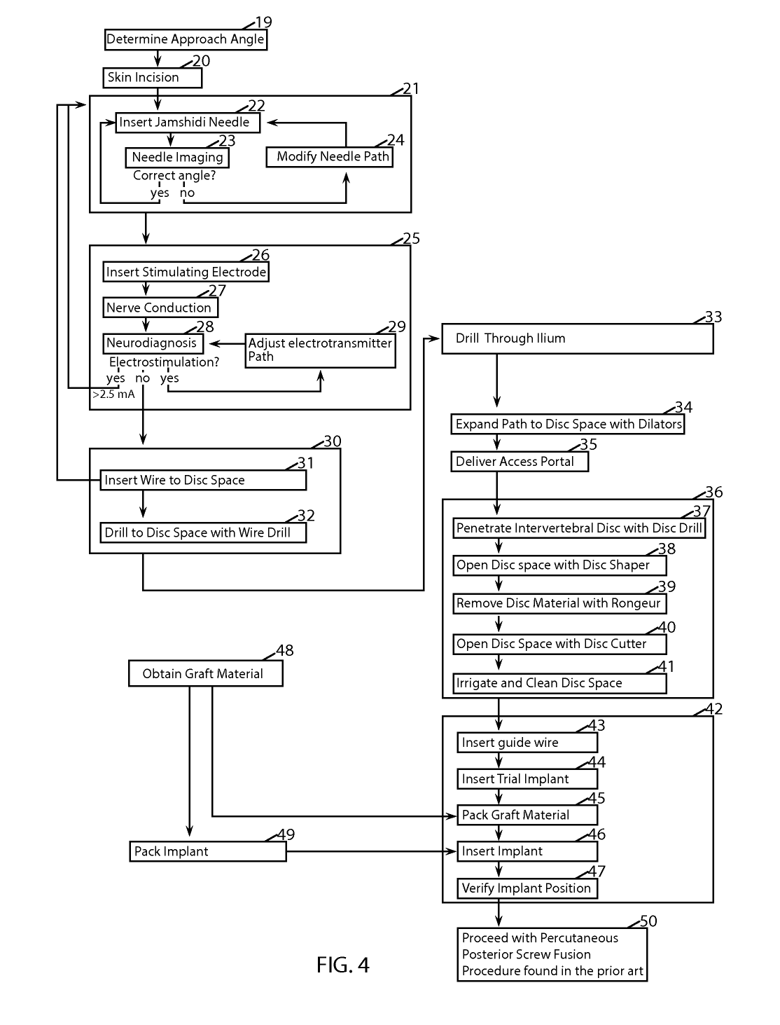

The general procedure for the preferred embodiment of the TOOLIF approach is illustrated in a flow chart in FIG. 4. After anesthesia, disinfection, and other standard procedures and practices related to surgery and/or spinal fusions known to persons having ordinary skill in the art, the medical practitioner undergoes the next step, Determine the Approach Angle 19 step, in the preferred embodiment of the invention. In the Determine the Approach Angle 19 step, the approach angle is determined by the medical practitioner. In certain embodiments, an approach angle is a relative angle of the ideal transiliac passage that allows various medical instruments 10 to access the L5-S1 intervertebral disc 1 space. In general, in the preferred embodiment of the invention, the step to Determine the Approach Angle 19 includes the following sub-steps: 1) evaluate the size of the patient (wherein such size consideration includes the height, waistline, waist size, weight, and/or other physical characteristics of the patient); 2) mark an area between six and nine inches from the midline of the patient; 3) insert the spinal needle through the skin; 4) image the surgical area in the lateral view and AP view with a bone-imaging device (such as, for example, a biplanar fluoroscope); and 5) adjust the approach angle of the spinal needle utilizing the images taken from such bone-imaging device.

In general, in the preferred embodiment of the invention, having a correct approach angle enables optimal access to the L5-S1 intervertebral disc 1 prior to drilling the transiliac aperture 9. With this embodiment in mind, it is explicitly intended in this application that a vertebral endplate, as is utilized in this application, includes but is not limited to an endplate of a sacrum, L5 lumbar vertebra, L4 lumbar vertebra, etc. In the Determine the Approach Angle 19 step, the medical practitioner utilizes a spinal needle. A spinal needle, in certain embodiments, includes instruments having a form that is linear, oblong, and sharp at its tip, and preferably having a radio-dense property. A spinal needle may be in the form of a needle or cannula of various gauges made of metal, for example, but is not limited to such embodiment. The radio-dense property for the spinal needle, in the preferred embodiment of the invention, enables a medical practitioner to determine the correct approach angle using images from a bone-imaging device during the Determine the Approach Angle 19 step.

In the preferred embodiment of the invention, the medical practitioner uses the size of the patient to determine the location of the initial entry point of the spinal needle. For instance, for a patient having a waist size greater than 38 inches, the initial entry point of the spinal needle is located at least 7 inches from the midline of such patient's body. In one embodiment of the invention, the initial entry point of the spinal needle is empirically determined. In the preferred embodiment of the invention, the medical practitioner adjusts the approach angle and/or potential incision site of the spinal needle such that the angle of entry of such spinal needle is parallel, or approximately 0°, relative to the plane of the L5-S1 intervertebral disc 1. Moreover, the medical practitioner adjusts the approach angle and/or potential incision site of the spinal needle such that the approach angle of such spinal needle is predicted to reach the center of the L5-S1 intervertebral disc space. In the preferred embodiment of the invention, during the Determine the Approach Angle 19 step, the approach angle and/or incision site of the spinal needle is adjusted and verified by referencing lateral-view and AP-view images taken by a bone-imaging device. In the preferred embodiment of the invention, such adjustments are made and verified by anticipating the path that the spinal needle would take if, for example, a hypothetical straight line was extended from the spinal needle to the L5-S1 intervertebral disc 1. By referencing a plurality of images taken by the bone-imaging device in the context of the spinal needle, and/or marking the path of such hypothetical line on an x-ray image from a lateral view, the medical practitioner is able to view and adjust the approach angle and/or potential incision site during the step to Determine the Approach Angle 19 in the preferred embodiment of the invention.

The Skin Incision 20 step is generally performed after the Determine the Approach Angle 19 step in the preferred embodiment of the invention. A skin aperture, in certain embodiments, is an opening through the skin. In certain embodiments of the invention, such skin aperture is created using instruments related to surgery and/or spinal fusions known to persons having ordinary skill in the art, such as for example, with a scalpel. In general, the Skin Incision 20 step creates the initial spinal needle, wherein such spinal needle is approximately the size of an implant and/or size of the instruments utilized during the TOOLIF approach, in the preferred embodiment of the invention. In one example, if an implant has a cross-sectional size of 12 mm, the incision in the Skin Incision 20 step is 12 mm. In general, the size of such incision to create the spinal needle is matched to the size of the instruments utilized during the TOOLIF approach as to minimize the size of such incision and/or minimize the invasiveness of such TOOLIF approach.

The Calibrate Ilium Approach 21 step, in the preferred embodiment of the invention, is in general, related to establishing an initial path through the Ilium. The advantage of incorporating the Calibrate Ilium Approach 21 step in the preferred embodiment of the invention is that it enables the medical practitioner to carefully refine the approach angle and/or approach path to create the transiliac passage. By carefully refining the approach angle and/or approach path during the Calibrate Ilium Approach 21 step, a medical practitioner can establish the approach angle and/or approach path to the L5-S1 intervertebral disc 1 space prior to the Drill Approach Path 33 step, thereby avoiding unnecessary drilling of the iliac bone in the preferred embodiment of the invention. By avoiding unnecessary drilling, the Calibrate Ilium Approach 21 step can reduce the overall invasiveness of the procedure and increase the speed at which the TOOLIF approach is completed, overall saving time and money as compared to other procedures found in the prior art.

In the preferred embodiment of the invention, the Calibrate Ilium Approach 21 step, in general, includes: 1) Insert the Jamshidi® Needle 22 step; 2) Needle Imaging 23 step; and 3) Modify Needle Path 24 step. The Insert the Jamshidi® Needle 22 step includes the insertion of a piercing tool, such as for example, but not limited to a Jamshidi® Needle that enables penetration through a bone structure. In the preferred embodiment of the invention, in such Insert the Jamshidi® Needle 22 step, the medical practitioner places a trephine needle such as a Jamshidi® Needle or other related device, wherein such device is a piercing tool that has a sharpened tip to penetrate bone, at an approach path and approach angle as ascertained by the Determine the Approach Angle 19 step. The medical practitioner further taps, i.e. strike lightly, the Jamshidi® Needle or other related device to partially penetrate the iliac bone.

In the preferred embodiment of the invention, following the Insert the Jamshidi® Needle 22 step, the Needle Imaging 23 step is performed to validate the approach angle and/or approach path of the Jamshidi® Needle or other related device, as such Jamshidi® Needle or other related devices traverses the ilium. In the Needle Imaging 23 step, the medical practitioner views the location and predicted trajectory of the Jamshidi® Needle or other related device within the ilium with respect to the L5-S1 intervertebral disc space, using images obtained from a bone-imaging device. In the preferred embodiment of the invention, the medical practitioner refers to a lateral view and/or AP view of TOOLIF approach area, such as for example, x-ray images of both a lateral view and/or AP view. If the medical practitioner determines that the Jamshidi® Needle or other related device has an ideal trajectory within the ilium that follows the ideal transiliac passage into the L5-S1 intervertebral disc 1 space, the medical practitioner returns to the Insert the Jamshidi® Needle 22 step. If the Jamshidi® Needle or other related device does not have the trajectory that follows the ideal transiliac passage into the L5-S1 intervertebral disc 1 space, the approach angle and/or approach path of such Jamshidi® Needle or other related device is altered during the Modify Needle Path 24 step. Once an adjustment to the Jamshidi® Needle or other related device is made during the Modify Needle Path 24 step, the medical practitioner continues with the Insert the Jamshidi® Needle 22 step wherein the medical practitioner may further tap, i.e. strike lightly the Jamshidi® Needle or other related device through the iliac bone. In this manner, in the preferred embodiment of the invention, the medical practitioner adjusts the approach path and the approach angle of the Jamshidi® Needle or other related device in the Calibrate Ilium Approach 21 step. In the preferred embodiment of the invention, the Calibrate Ilium Approach 21 step is completed once the Jamshidi® Needle or other related device traverses the ilium, effectively creating an initial path for the transiliac passage.

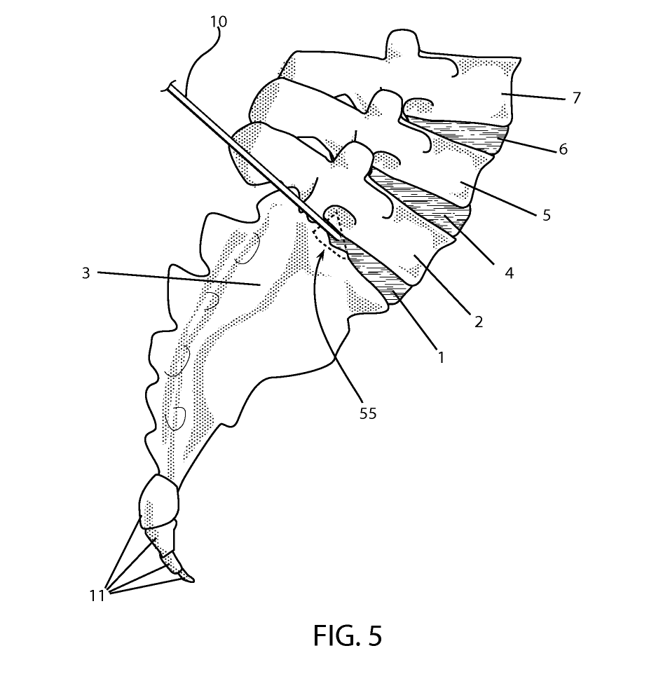

In the preferred embodiment of the invention, following the Calibrate Ilium Approach 21 step, the Medical practitioner proceeds with the Calibrate Kambin Approach 25 step. In the preferred embodiment of the invention, the Calibrate Kambin Approach 25 step establishes an approach path that avoids nerves associated with the spine. More specifically, as illustrated in FIG. 5, the preferred embodiment of the invention describes access to the L5-S1 intervertebral disc space through Kambin's Triangle 55. Nerves associated with Kambin's Triangle include the S1 Traversing Nerve Root, the L5 Exiting Nerve Root, and other related nerves located proximal to the spine. The S1 Traversing Nerve Root, in the context of the TOOLIF approach, refers to the S1 nerve root, and the L5 Exiting Nerve Root refers to the L5 nerve root. In the preferred embodiment of the invention, the Calibrate Kambin Approach 25 step in general includes: 1) Insert Stimulating Electrode 26 step; 2) Nerve Conduction 27 step; 3) Neurodiagnosis 28 step and 4) Adjust Stimulating Electrode Path 29 step.

Once the initial path for the transiliac passage is created by the Calibrate Ilium Approach 21 step, the medical practitioner inserts a stimulating electrode through the spinal needle and the transiliac passage during the Insert Stimulating Electrode 26 step in the preferred embodiment of the invention. In the preferred embodiment of the invention, the guide wire includes a device having a degree of rigidity to enable penetration through tissue with applied force. In general, in the preferred embodiment, a guide wire is typically used during surgical procedures requiring multiple instruments to follow a distinct path to the surgical site. In one example associated with the preferred embodiment, a guide wire is used as a placeholder, wherein larger medical instruments can access the surgical site by following and/or traversing over such guide wire while ensuring that such larger medical instrument does not stray from the distinct path. Therefore, by using the guide wire, potential damage by medical instruments to regions outside of the approach path, including other tissue, nerves, organs, and other regions of the body, is avoided. In varying embodiments, the guide wire may be in the form of an electro-conductive wire or probe, such as a stimulating electrode. In an embodiment of the invention, a stimulating electrode is a type of device having a degree of rigidity further including an electro-conductive wire or probe. In certain embodiments, a stimulating electrode may be used with or interchangeably with a guide wire, or have characteristics of a guide wire during a Calibrate Kambin Approach 25 Step, in certain embodiments, a stimulating electrode associated with the Calibrate Kambin Approach 25 step may have electroconductive properties. In an embodiment of the invention, the guide wire is a wire having a diameter between 1.0 and 3.0 mm.

In the preferred embodiment of the invention, the medical practitioner further utilizes an electrosurgical instrument in association with a stimulating electrode or electrically stimulating guide wire during the Calibrate Kambin Approach 25 step, as well as in the Nerve Conduction 27 step. An electrosurgical instrument, in the preferred embodiment, is a current-generating device having a current-generating module, current measurement module, electro stimulating electrode and elecroreceiving electrode, known to those skilled in the art. In general, in the Nerve Conduction 27 step in the preferred embodiment, the electrostimulating electrode from the electrosurgical instrument is attached to the guide wire, in effect, enabling the current from the electrosurgical instrument to flow through the guide wire to reach the surgical site. Moreover, the electroreceiving electrode is attached to a distal portion of the body, such as for example, in proximity to the gastrocnemius muscle, or in proximity to the dorsal flexors of the foot. By placing the electroreceiving electrode in these locations, and further stimulating the electrode passing through Kambin's Triangle, a medical practitioner can navigate through Kambin's Triangle, and avoid the S1 Traversing Nerve Root by receiving feedback from the gastrocnemius muscle, or avoid the L5 Exiting Nerve Root by receiving feedback from the dorsal flexors of the foot. In certain embodiments, a needle linked with the electroreceiving electrode or a plurality of electroreceiving electrodes may penetrate skin and inserted in the vicinity or within such muscles. Yet in certain embodiments, an electroreceiving electrode may be placed in the vicinity, for example, on an exterior surface of the skin near such muscles. In the preferred embodiment, a patient may be injected with a non-paralytic anesthetic into, and/or around the gastrocnemius muscle and/or the dorsal flexors. In the preferred embodiment of the invention, by activating the current-generating module, an electric current is essentially introduced at the guide wire. If such guide wire is in close proximity to a nerve, an electric current travels through the nerve or nerves, and is transmitted through the electroreceiving electrode and is further detected by the current measurement module. In effect, the electrosurgical instrument is utilized to determine the approach path while avoiding nerves associated with the spine, more specifically, the S1 Traversing Nerve Root, and the L5 Exiting Nerve Root.

In the preferred embodiment of the invention, during the Nerve Conduction 27 step, the medical practitioner adjusts the current of the electrosurgical instrument as to refine the approach path. In general, the current of the electrosurgical instrument is adjusted while the guide wire traverses Kambin's Triangle 55 and accesses the L5-S1 intervertebral disc 1. The inventor has observed that a current of greater than 20 mAmps results in a spread of the electrical current and possible false positive readings regarding the location of the nerves in relation to the guide wire. Therefore, in the preferred embodiment of the invention, the medical practitioner adjusts the electrosurgical instrument to emit 14 mAmps to the guide wire. Other embodiments of the invention allow for the initial current to be between 10 mAmps and 20 mAmps. As the medical practitioner passes the guide wire in proximity to the S1 Traversing Nerve Root and the L5 Exiting Nerve Root, the current of the electrosurgical instrument is decreased to between 7.5 mAmps and 5 mAmps in the preferred embodiment. During the Neurodiagnosis Step 28, the medical practitioner adjusts the current passing from an stimulating electrode, and evaluates the observable stimulation of the electroreceiving electrode in the preferred embodiment of the invention. When the current is adjusted to between 7.5 mAmps and 5 mAmps, the lack of observable stimulation at the electroreceiving electrode indicates that the guide wire is passing through an area that is not occupied by nerves associated with the spinal column. On the other hand, an observable stimulation at the electroreceiving electrode at the gastrocnemius muscle, or in proximity to the dorsal flexors of the foot, indicates that the guide wire is potentially in the close vicinity of the S1 Traversing Nerve Root or L5 Exiting Nerve Root respectively, and indicates to the medical practitioner to adjust the electrotransmitter path as to avoid damaging or injuring such nerves in subsequent steps. In the preferred embodiment of the invention, the medical practitioner adjusts and further establishes the approach path of the guide wire during the Calibrate Kambin Approach step 25 until the guide wire reaches the L5-S1 intervertebral disc 1. The passage of the guide wire to the L5-S1 intervertebral disc 1 is further confirmed by reducing the current of the electrosurgical instrument to 2 mAmps, and verifying that no observable stimulation is present at the electroreceiving electrode. In general, in the preferred embodiment of the invention, the medical practitioner adjusts the current of the electrosurgical instrument between 20 mAmps and 2 mAmps during the Calibrate Kambin Approach step 25.

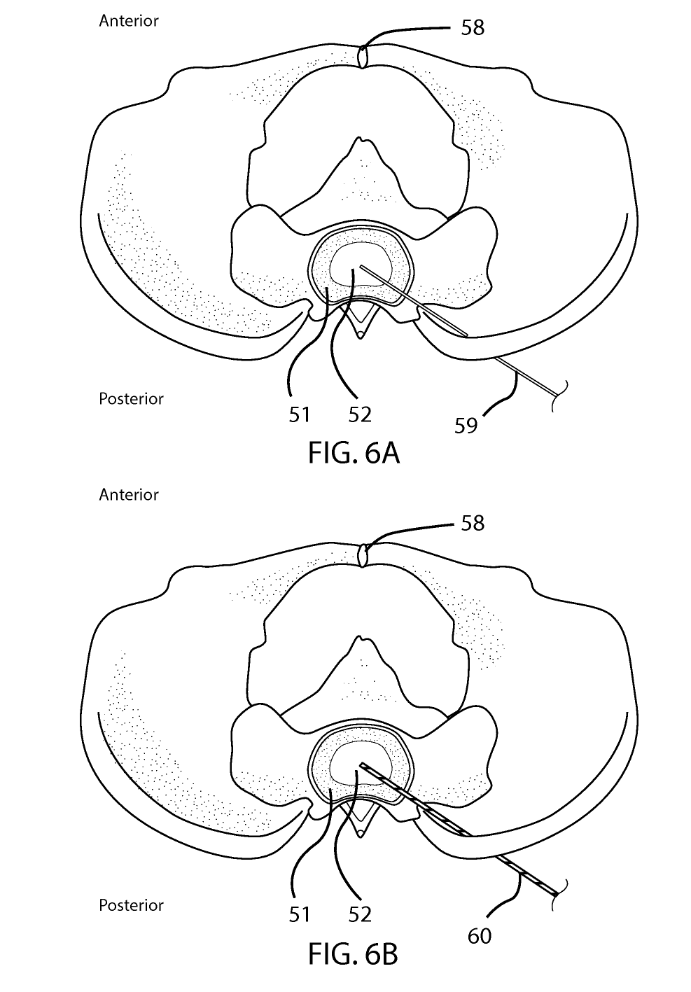

The Establish Approach Path step 30, in the preferred embodiment of the invention, includes a number of sub-steps that establishes the transiliac passage. In the preferred embodiment of the invention, during the Insert Wire to Disc Space step 31, the medical practitioner pushes the guide wire to the center of the L5-S1 intervertebral disc 1. In effect, the guide wire is pushed through the Anulus Fibrosis 51 and into the Nucleus Pulposus 52 such that the end of the guide wire reaches the center of the L5-S1 intervertebral disc space as illustrated in FIG. 6A. In the preferred embodiment of the invention, the location of the guide wire 59 is confirmed by, and/or adjusting by referencing a bone-imaging device, such that the end of such guide wire reaches 50% of the distance into the intervertebral disc space from the anterior view, and 50% of the distance into the intervertebral disc space from the lateral view, as illustrated in FIG. 6A. In the preferred embodiment of the invention, once the guide wire reaches the L5-S1 intervertebral disc 1, the guide wire effectively establishes transiliac passage from the exterior of the body to the L5-S1 intervertebral disc 1 for medical instruments to follow such transiliac passage.

In the preferred embodiment of the invention, a medical practitioner uses a wire drill to secure the path of the transiliac passage. A wire drill, is a mechanical drill that has the ability to follow the path of a guide wire. A wire drill found in the prior art, includes a drill bit with an opening that enables such wire drill to slide over the guide wire. In the preferred embodiment of the invention, the wire drill used during the TOOLIF approach further includes a straight, non-bending drill bit, wherein such drill bit is between 1.5 mm and 4 mm in diameter.

During the Drill to Disc Space with Wire Drill Step 32, in the preferred embodiment of the invention, the medical practitioner follows the path established by the guide wire during the Insert Wire to Disc Space step 31, to establish straight path to the L5-S1 intervertebral disc space. As illustrated in FIG. 6B, the wire drill 60 follows the path of the guide wire 59 to establish a direct path from the exterior of the patient's body to the L5-S1 Intervertebral Disc Space. The advantage of using the disc drill at this point of the TOOLIF approach is that the straight, non-bending tip of the disc drill establishes a direct path from the exterior of the body to the L5-S1 intervertebral disc space.

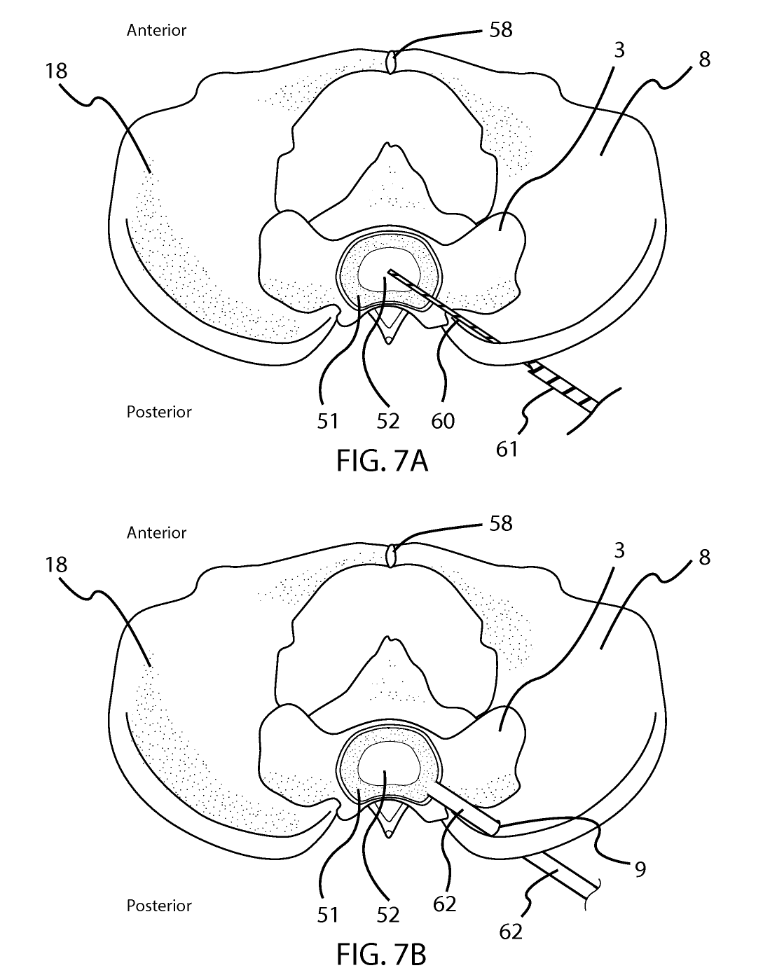

In the preferred embodiment of the invention, the Drill Through Ilium Step 33 further widens the transiliac passage. In general, in the preferred embodiment of the Drill through Ilium Step 33 follows the transiliac passage previously established by the Calibrate Ilium Approach Step 21, the Calibrate Kambin Approach Step 25, and the Drill to Disc Space with Wire Drill Step 32. The bone drill 61 utilized during the preferred embodiment of the Drill Through Ilium Step 33 has a drill bit further having hole, such that such drill bit can follow the wire drill used during the Drill to Disc Space with Wire Drill Step 32, as illustrated in FIG. 7A. The key differentiating aspect of this step, as compared to prior art methods, is the approach angle and approach path to access the L5-S1 intervertebral disc 1 is established prior to drilling a hole through the ilium. By following such approach path and approach angle, the preferred embodiment of the invention enables the medical practitioner to drill through the ilium just once, to create the transiliac passage 9, effectively solves the problem of unnecessary trial and error drilling. Furthermore, one of the key aspects of the Drill through Ilium Step 33 in the preferred embodiment of the invention is to create an aperture through the ilium that can be subsequently accessed with a number of medical instruments of a certain diameter. In the preferred embodiment of the invention, the aperture created by the bone drill 61 during the Drill through Ilium Step 32 is between 7 mm and 13 mm.

In the preferred embodiment of the invention, the medical practitioner further enlarges the approach path to the L5-S1 intervertebral disc 1 using a series of dilators, during the Expand Path to Disc Space with Dilators step 34. In certain embodiments, dilators are a series of cylinder-like elongated instruments, wherein the each dilator has a diameter that is successively larger than another, known to persons skilled in the art. In the preferred embodiment of the invention, a dilator has an opening that enables a dilator to slide over a guide wire or other dilators, such that the dilators follow a guide wire or other dilators to expand the opening of the transiliac passage. The medical practitioner can therefore utilize successively larger dilators through the transiliac passage to stretch the tissue surrounding the guide wire, and to expand the opening of the transiliac passage. In the preferred embodiment of the invention, the series of successive dilators have diameters that are 2.5 mm, 4.5 mm, 6.5 mm, and 10 mm in length. In other embodiments of the invention, the number of dilators used during the Expand Path to Disc Space with Dilators step 34 is between two and five, having diameters that are in the range of 2 mm and 10 mm. In the preferred embodiment of the invention, the medical practitioner increases the diameter of the transiliac passage to 10 mm in diameter, as to access the L5-S1 intervertebral disc 1 with the appropriate medical instruments used during the Intervertebral Disc Removal step 36.

The present inventor has discovered that despite the presence of a transiliac aperture and/or transiliac passage having a diameter of 10 mm or less, the bone and/or tissue will give way several millimeters during the steps associated with placement of an implant into and through the transiliac aperture and/or transiliac passage in order to accommodate the necessarily larger sized implants to enable effective interbody fusion. In embodiments of the invention therefore, medical practitioner may place an up to 14 mm implant at L5-S1 through a 10 mm transiliac aperture and/or transiliac passage after removing the access portal by tamping the implant or bodies associated with placement of the implant during steps associated with the method described herein. Said tamping of the implant or bodies associated with placement of the implant forces the implant through the transiliac aperture and/or transiliac passage despite the potentially larger diameter of the implant as compared to the transiliac aperture and/or transiliac passage. In this manner, the medical practitioner may tamp the implant through the osseous opening for placement into the disc space. In an embodiment, the guide wire may optionally assist in the placement of the implant, however in the preferred embodiment of the invention, the guide wire is not necessary for processes associated with guiding the implant for placement into the disc space. Thus, the medical practitioners may optionally remove the guide wire prior to the placement of the implant into and/or through the transiliac aperture and/or transiliac passage.

In the preferred embodiment of the invention, the Deliver Access Portal Step 35 establishes a path that enables passage of Disc Instruments to the L5-S1 intervertebral disc 1. In certain embodiments, an access portal is a type of dilator that has surface features that provide traction on or between bone structures. An access portal, in the preferred embodiment of the invention, is tapped, i.e. struck by a blunt instrument by the medical practitioner, in order to establish a stably placed, tubular opening from the exterior of the body to the L5-S1 intervertebral disc space with such access portal 62, as illustrated in FIG. 7B. In the preferred embodiment of the invention, the medical practitioner taps the access portal 62 such that the distal end of the access portal is secured, by compression of the access portal 62 between the 5^{th} Lumbar Vertebra 2 and the Sacrum 3. In the preferred embodiment of the invention, the access portal 62 is further secured to the ilium by surface features, such as an enlarged circumference, and/or ridges, that is secured to the ilium aperture through compression of the access portal within such ilium aperture. In general, the access portal enables the medical practitioner to guide, maneuver, or otherwise utilize medical instruments during the Intervertebral Disc Removal step 36.

In the preferred embodiment of the invention, the Intervertebral Disc Removal step 36 has a number of sub-steps for the purposes of removing disc material between the L5 and S1 vertebrae. In the preferred embodiment of the invention, the Intervertebral Disc Removal step 36 includes the following sub-steps: 1) Penetrate Intervertebral Disc with Disc Drill step 37; 2) Open Disc Space with Disc Shaper 38; 3) Remove Disc Material with Rongeur 39; 4) Open Disc space with Disc Cutter 40; and 5) Irrigate and Clean Disc Space 41. While the preferred embodiment of the invention during the Intervertebral Disc Removal step 36, includes these steps as described above in the present paragraph, other embodiments of the invention may include such steps in a different sequence. In certain embodiments, a disc drill is an instrument having a rotating tip having acutely angled edges for penetrating the annulus fibrosus and nucleus pulposus. In certain embodiments, a disc shaper, is a tool that typically includes a flattened end that helps a medical practitioner to open an intervertebral disc space by scraping the disc material. In certain embodiments, a rongeur, also known as a Kerrison rongeur, includes a grabbing tip that enables a medical practitioner to take a hold of pieces of disc material and remove such pieces from the intervertebral disc space to the exterior of the body. In certain embodiments, a disc cutter is a tool having a sharpened edge that helps a medical practitioner to open an intervertebral disc space by scraping the disc material from the endplates of the vertebral bodies. A disc drill, disc shaper, rongeur, and disc cutter, are commonly used items during spinal fusion surgery known to persons having skill in the art. It will be appreciated by those skilled in the art that a number of tools may be used to clean a vertebral disc material from a vertebral disc space. In the preferred embodiment of the invention, the Intervertebral Disc Removal step 36 removes the L5-S1 intervertebral disc material such that the intervertebral disc space is subsequently filled with the implant and/or graft material.

In varying embodiments of the invention and methods described herein, the graft material may be harvested from iliac bone, for example, as shavings from drilling through such ilium to create a transiliac aperture. When the medical practitioner removes the bone drill associated with the creation of the transiliac aperture, bone graft by-product is removed within the threads of the drill, which may subsequently be repurposed during a later step associated with the methods described herein as graft material. It will be appreciated that bone used for certain steps may be either autograft, allograft, or a combination, and may further include stem cells, demineralized bone matrix, bone glue, etc. Bone graft material is a preferred substance to use in association with spinal fusion, and this is utilized in association with the preferred embodiment of the invention described herein.

Effective removal of existing disc material facilitates bone fusion of the L5 Vertebra and the S1 Vertebra with the graft material. Therefore, in the preferred embodiment of the invention, the Intervertebral Disc Removal step 36 further includes a Irrigate and Clean Disc Space 41, wherein a sterile liquid, such as water, saline solution, or buffer solution, is placed in the intervertebral disc space, and disc material is further removed by suspending such disc material in such sterile liquid and vacuuming out such suspension, effectively removing residual disc material from the intervertebral disc space.

In the preferred embodiment of the invention, the Intervertebral Disc Space Implantation step 42 includes a number of sub-steps for the purposes of further placing an implant in the L5-S1 intervertebral disc space for spinal fusion. In the preferred embodiment of the invention, the Intervertebral Disc Space Implantation step 42 includes the following sub-steps: 1) Insert guide wire for Implant 43; 2) Insert Trial Implant 44; 3) Pack Graft Material 45; 4) Insert Implant 46; and 5) Verify Implant position 47. In the preferred embodiment of the invention, the Insert guide wire for Implant step 43 includes placing the guide wire through the Access portal as to establish the path for the implant. An implant, also known as a cage, and may be made of a number of materials such as carbon fiber or polyetheretherkeyone (PEEK) known to those having skill in the art. The implant has an orifice or a plurality of orifices that accommodate graft material, wherein a medical practitioner fills the orifice or plurality of orifices of the implant with graft material during the Pack Implant step 49. In certain embodiments, the implant, in general, further has a radiodense material, for instance a rod, to verify the position and orientation of such implant in a radiographic image. In one embodiment of the invention, the implant used during the TOOLIF approach has a hole or a plurality of holes in which such implant follows a guide wire through for delivery to the L5-S1 intervertebral disc space. In another embodiment of the invention, a medical practitioner pushes the implant through the access portal for delivery of such implant to the L5-S1 intervertebral disc space. In general, in the preferred embodiment of the invention, the procedures performed during the Intervertebral Disc Space Implantation step 42 are performed by access through the access portal and/or the transiliac passage.

Implants of a number of various sizes are available in the prior art, wherein the height of the implant varies such that an ideal implant is chosen for use between two vertebral discs. Such implants typically has a height range of 9 mm to 14 mm. In general, in the preferred embodiment of the invention, the implant size for an TOOLIF approach is in general, larger than the intervertebral disc space of the patient that is undergoing such TOOLIF approach as to ensure a snug fit. Following the Insert guide wire for Implant step 43, a medical practitioner places a trial implant or a plurality of trial implants during the Insert Trial Implant step 44. In the preferred embodiment of the invention, a number of trial implants may be placed in the L5-S1 intervertebral disc space in order to determine the correct implant size for spinal fusion. In the preferred embodiment of the invention, the correct implant size is determined by inserting successively larger implant into the L5-S1 intervertebral disc space. For example, a medical practitioner inserts a 10 mm implant into an intervertebral disc space of a patient. The present inventor has discovered that if there is little to no resistance of entry of such implant into such intervertebral disc space, such implant is considered to be smaller than ideal. Thus such smaller than ideal implant in the preferred embodiment described herein is not utilized for spinal fusion. However, in other embodiments, a smaller than ideal implant is used to facilitate spinal fusion. In another example, a medical practitioner may insert a 12 mm implant into the same intervertebral disc space of a patient used in the previous example. If the resistance of entry of a 12 mm implant into such intervertebral disc space requires a force that permits spinal fusion of the vertebrae, then such implant is used. In general, medical practitioners having skill in the art are knowledgeable, through their experience, in understanding the ideal implant size necessary to achieve spinal fusion. In this manner, during the preferred embodiment of the Insert Trial Implant step 44, the medical practitioner determines the correct implant for the spinal fusion.

In the preferred embodiment of the Pack Graft Material step 45, the medical practitioner places graft material into the intervertebral disc space. In certain embodiments, graft material may include morselized autograft or allograft bone matter. In general, in the preferred embodiment of the invention, the graft material is generated during the Obtain Graft Material step 48. During the Obtain Graft Material step 48 a medical practitioner breaks down bone material such that such bone material is placed in the L5-S1 intervertebral disc space to promote spinal fusion. In an embodiment of the invention, the graft material may include stem cells and/or platelet aggregates in whole or in part. The present inventor has discovered that the incorporation of stem cells and/or platelet aggregates facilitates increased healing and thus an improved clinical result. During the Pack Graft Material step 45 the medical practitioner pushes the graft material such that the space between the vertebral discs, previously opened by removal of the disc material during the Intervertebral Disc Removal step 36, is filled with the graft material. During the Insert Implant Step 46 the implant, further having graft material placed within an orifice of plurality of orifices of such implant, is further placed into the intervertebral disc space. In this manner, the implant helps to preserve the spacing of the intervertebral disc space, while the graft material surrounding the implant facilitates bone fusion of the vertebrae. Furthermore, the placement of the implant is verified during the Verify Implant Position step 47. In the preferred embodiment of the invention, the implant location, rotation, or other information relative to the L5 vertebra and the S1 Vertebra is determined by imaging the surgical area in the lateral view and AP view with a bone-imaging device (such as, for example, a biplanar fluoroscope). In the preferred embodiment of the invention, if the implant is incorrectly placed, the medical practitioner assess further corrects the placement of the implant.

Once the implant location, rotation, or other information relative to the L5-S1 intervertebral disc space is verified, the medical instruments are removed from the patient. General procedures to close the incision previously created during the Skin Incision step 20 are performed as to close the spinal needle.

In the preferred embodiment of the invention, the medical practitioner further proceeds with a Percutaneous Posterior Fixation Procedure 50. In general, the Fixation Procedure includes procedures known to persons having skill in the art that stabilizes two or more vertebrae. One example of a Percutaneous Posterior Fixation Procedure includes a Pedicle Screw Fixation Procedure, wherein a structure keeps one or more vertebrae stabilized, by affixing such structure to the pedicles of such vertebrae. In general, the Fixation Procedure essentially immobilizes the vertebrae such that the spinal fusion occurs during healing of the patient.

The illustrations of arrangements described herein are intended to provide a general understanding of the structure of various embodiments, and they are not intended to serve as a complete description of all the elements and features of apparatus and systems that might make use of the structures described herein. Many other arrangements will be apparent to those of skill in the art upon reviewing the above description. Other arrangements may be utilized and derived therefrom, such that structural and logical substitutions and changes may be made without departing from the scope of this disclosure. Figures are also merely representational and may not be drawn to scale. Certain proportions thereof may be exaggerated, while others may be minimized. Accordingly, the specification and drawings are to be regarded in an illustrative rather than a restrictive sense.

The preceding description has been presented with reference to various embodiments. Persons skilled in the art and technology to which this application pertains will appreciate that alterations and changes in the described structures and methods of operation can be practiced without meaningfully departing from the principle, spirit and scope.

The present systems, methods, means, and enablement are not limited to the particular systems, and methodologies described, as there can be multiple possible embodiments, which are not expressly illustrated in the present disclosures. It is also to be understood that the terminology used in the description is for the purpose of describing the particular versions or embodiments only, and is not intended to limit the scope of the present application.

Some embodiments, illustrating its features, will now be discussed in detail. The words “comprising,” “having,” “containing,” and “including,” and other forms thereof, are intended to be equivalent in meaning and be open ended in that an item or items following any one of these words is not meant to be an exhaustive listing of such item or items, or meant to be limited to only the listed item or items. It must also be noted that as used herein and in the appended claims, the singular forms “a,” “an,” and “the” include plural references unless the context clearly dictates otherwise. Although any methods, and systems similar or equivalent to those described herein can be used in the practice or testing of embodiments, the preferred methods, and systems are now described. The disclosed embodiments are merely exemplary

I claim:

1. A method of accessing the area between a first vertebral endplate and a second vertebral endplate and traversing an opening through an ilium for the purposes of bone fusion, comprising the steps of:
placing a surgical instrument through an ilium to guide a path to the area of a vertebral disc;
creating a transiliac aperture along said path;
removing a vertebral disc through said transiliac aperture; and
filling the space previously occupied by said vertebral disc with material placed through said transiliac aperture.

2. The method of claim 1, wherein said vertebral disc is located between a L5 lower endplate and a S1 upper endplate.

3. The method of claim 1, further comprising identifying a non-disruptive path prior to the placing a surgical instrument step.

4. The method in claim 3, wherein said identifying takes place by utilizing an electrosurgical instrument.

5. The method in claim 4, wherein said electrosurgical instrument enables access through nerve structures.

6. The method in claim 1, wherein the filling step comprises the placement of an implant and bone graft material.
